(19) United States
(12) Patent Application Publication
(10) Pub. No.: US 2025/0213293 A1
(43) Pub. Date: Jul. 3, 2025

(54) A CATHETER FOR USE IN A SYSTEM FOR FORMING A FISTULA

(71) Applicant: CLEARSTREAM TECHNOLOGIES LIMITED, Enniscorthy, County Wexford (IE)

(72) Inventors: Nicola Broaders, Enniscorthy, County Wexford (IE); John O'Shea, Enniscorthy, County Wexford (IE); Michael Whelan, Enniscorthy, County Wexford (IE)

(21) Appl. No.: 18/853,126

(22) PCT Filed: Apr. 1, 2022
(86) PCT No.: PCT/EP2022/058802

§ 371 Date: Sep. 30, 2024

(51) Int. Cl. A61B 18/14; A61B 17/00; A61B 18/00

(52) U.S. Cl. CPC A61B 18/1492; A61B 2017/0088; A61B 2018/00083; A61B 2018/00107; A61B 2018/00428; A61B 2018/00577

(57) ABSTRACT

A catheter for use in a system for forming a fistula between two vessels. The catheter comprises a catheter shaft, and a split backstop coupled to the catheter shaft and configured to receive a fistula-forming electrode. The split backstop has a proximal section and a distal section. The proximal section and the distal section are moveable relative to each other.

### TECHNICAL FIELD

The present disclosure relates to a catheter for use in a system for forming a fistula between two vessels and a system for forming a fistula between two vessels.

### BACKGROUND

A fistula denotes a passageway formed between two internal organs. Forming a fistula between two blood vessels can have one or more functions. For example, the formation of a fistula between an artery and a vein may provide access to the vasculature for haemodialysis patients. Specifically, forming a fistula between an artery and a vein allows blood to flow quickly between the vessels while bypassing the capillaries. Needles, catheters, or other cannulas may then be inserted into the blood vessels near the fistula to draw blood from the circulatory system, pass it through a dialysis machine, and return it to the body. The quickened flow provided by the fistula may provide for effective haemodialysis.

In other instances, a fistula may be formed between two veins to form a veno-venous fistula. Such a veno-venous fistula may be used to treat portal venous hypertension. Specifically, cirrhosis or other liver diseases may cause increased resistance to flow from the portal veins draining from the intestine to the liver. This increased resistance may cause massive dilation of blood vessels, which may rupture spontaneously. To help prevent this, a fistula may be formed between a portal vein and one of the major branches, thereby lowering venous pressure in the portal vein

US 2012/0302935 A1 and US 2017/0202616 A1 disclose systems and methods for forming a fistula using an endovascular approach. These systems comprise a first catheter having a housing with an electrode and a second catheter having a non-conductive ceramic backstop. However, due to the rigid nature of the ceramic backstop, it can be difficult to navigate the second catheters through tortuous vessel anatomy, such as the anterior tibial vessel, for example.

In view of the above, there is a need in the art for a new catheter system which can more easily navigate tortuous vessel anatomy.

### SUMMARY

In a first aspect of the present disclosure, there is provided a catheter for use in a system for forming a fistula between two vessels. The catheter comprises a catheter shaft, and a split backstop coupled to the catheter shaft and configured to receive a fistula-forming electrode. The split backstop has a proximal section and a distal section. The proximal section and the distal section are moveable relative to each other.

In some embodiments, this may result in a catheter which can more easily navigate tortuous vessel anatomy.

Throughout this disclosure, the term ‘fistula’ is used to denote a connection or passageway.

The proximal section and the distal section may be tiltable relative to each other.

Throughout this disclosure, the term ‘tilt’ is used to denote the motion of forming an angle between two elements whilst maintaining a point of connection.

The proximal section and the distal section may together form a recessed portion.

In some embodiments, this may result in better compression of the vessel walls and therefore better fistula formation.

The recessed portion may have a shape complimentary to the fistula forming electrode.

In some embodiments, this may result in better engagement of the electrode with the backstop and therefore better fistula formation.

The recessed portion may have a U-shape.

The recessed portion may have a concave shape.

The split backstop may be non-conductive.

In some embodiments, this may prevent arcing between the electrode and the backstop and results in better fistula formation.

The split backstop may be, at least partly, made from a ceramic material.

In some embodiments, this may allow the backstop to better withstand the heat and plasma generated by the electrode.

The distal end of the proximal section may have a rounded shape.

In some embodiments, this may allow the proximal section to better tilt relative to the distal section and vice versa.

The proximal end of the distal section may have a rounded shape.

In some embodiments, this may allow the distal section to better tilt relative to the proximal section and vice versa.

The proximal section and the distal section may be connected through a spring.

In some embodiments, this may provide greater flexibility to the backstop to allow it to better navigate tortuous vessel anatomy.

The spring may be insulated.

In some embodiments, this may prevent arcing between the electrode and the spring.

The proximal section and/or the distal section may comprise a bore for accommodating the spring.

In some embodiments, this may result in a more secure fit for the spring.

The spring may be a coil spring.

In some embodiments, this may allow for longitudinal as well as tilting movement of the distal section relative to the proximal section.

The spring may be a leaf spring.

In some embodiments, this may prevent longitudinal movement while allowing tilting movement of the distal section relative to the proximal section and vice versa.

Throughout this disclosure, the term ‘leaf spring’ is used to denote a flexible strip of material which can be bent but will regain its original shape when released.

The proximal section and the distal section may not be in direct contact.

In some embodiments, this may allow for a greater tilting movement of the distal section relative to the proximal section and vice versa.

The proximal section and the distal section may be connected through a ball and socket joint.

In some embodiments, this may provide greater flexibility to the backstop to allow it to better navigate tortuous vessel anatomy.

The ball and socket joint may comprise a ball disposed within a socket of the proximal section or the distal section, and a shaft attached to the ball.

The shaft may be fixed to the distal section if the socket is disposed in the proximal section, or the shaft may be fixed to the proximal section if the socket is disposed in the proximal section.

The ball and shaft may be made from a ceramic material.

In some embodiments, this may allow it to better withstand the high temperature and plasma generated by the electrode.

The ball and shaft may be coated with an insulating material.

The proximal section and the distal section may not be in direct contact.

In some embodiments, this may allow for a greater tilting movement of the distal section relative to the proximal section and vice versa.

The proximal section and the distal section may be connected through a connecting rod.

In some embodiments, this may provide flexibility to the backstop to allow it to better navigate tortuous vessel anatomy.

In some embodiments, this may further allow the backstop to have a small diameter and low profile to more easily advance through smaller vessels.

The proximal section and/or the distal section may comprise a bore for accommodating the connecting rod.

In some embodiments, this may result in a more secure fit of the connecting rod.

The connecting rod may be secured to the proximal and/or distal section with an adhesive.

In some embodiments, this may result in a more secure fit of the connecting rod.

The proximal section and the distal section may be in contact with each other.

In some embodiments, this may result in a more structurally robust backstop.

The connecting rod may comprise a polyimide tube.

The connecting rod may comprise a nitinol wire.

The nitinol wire may comprise an outer layer of insulating material.

In some embodiments, this may better protect the nitinol wire from the heat and plasma generated by the electrode and prevent arcing between the electrode and the nitinol wire.

The catheter may further comprise a set of proximal magnets, disposed proximally of the split backstop.

The catheter may further comprise a set of distal magnets, disposed distally of the split backstop.

In some embodiments, this may allow the catheter to easily and accurately align with a second catheter comprising an electrode.

In a second aspect of the present disclosure, there is provided a system for forming a fistula. The system comprises a first catheter according to any of the above embodiments, and a second catheter comprising a housing and a fistula-forming electrode extending radially from the housing.

In some embodiments, this may result in a system which can more effectively form a fistula between two vessels.

The fistula-forming electrode may be disposed at least partially within the housing.

The fistula-forming electrode may comprise a distal portion, a proximal portion and an intermediate portion therebetween for contacting a vessel wall and forming the fistula.

The intermediate portion may have a convex shape.

In some embodiments, this may result in better fistula formation.

The electrode may comprise a ribbon wire.

In some embodiments, this may result in better fistula formation.

The first catheter and the second catheter may comprise one or more sets of magnets positioned to align the fistula-forming electrode with the split backstop.

The system may further comprise a distal set of magnets disposed on the second catheter, distally of the housing.

The system may further comprise a proximal set of magnets disposed on the second catheter, proximally of the housing.

In some embodiments, this may allow for easy and accurate alignment of the electrode with the backstop in the vessel.

The housing may be, at least partly made from a ceramic material.

The system may further comprise an RF energy generator for supplying energy to the electrode.

### BRIEF DESCRIPTION OF THE DRAWINGS

To enable better understanding of the present disclosure, and to show how the same may be carried into effect, reference will now be made, by way of example only, to the accompanying drawings, in which:

FIG. 1 shows a system having a first and second catheter for forming a fistula between two vessels.

FIG. 2 shows the system of FIG. 1 disposed in a blood vessel system.

FIG. 3 shows a side view of a first catheter for use in a system for forming a fistula, according to the present disclosure.

FIG. 4 shows a side view of an alternative embodiment of a first catheter for use in a system for forming a fistula, according to the present disclosure.

FIG. 5A shows a side view of another alternative embodiment of a first catheter for use in a system for forming a fistula, according to the present disclosure.

FIG. 5B shows an isometric view of the first catheter of FIG. 5A.

FIG. 5C shows the first catheter of FIG. 5A inside a vessel.

FIG. 6A shows another alternative embodiment of a first catheter for use in a system for forming a fistula, according to the present disclosure.

FIG. 6B shows an isometric view of a portion of the backstop of the first catheter of FIG. 6A.

FIG. 7 shows a side view of another alternative embodiment of a first catheter for use in a system for forming a fistula, according to the present disclosure.

### DETAILED DESCRIPTION

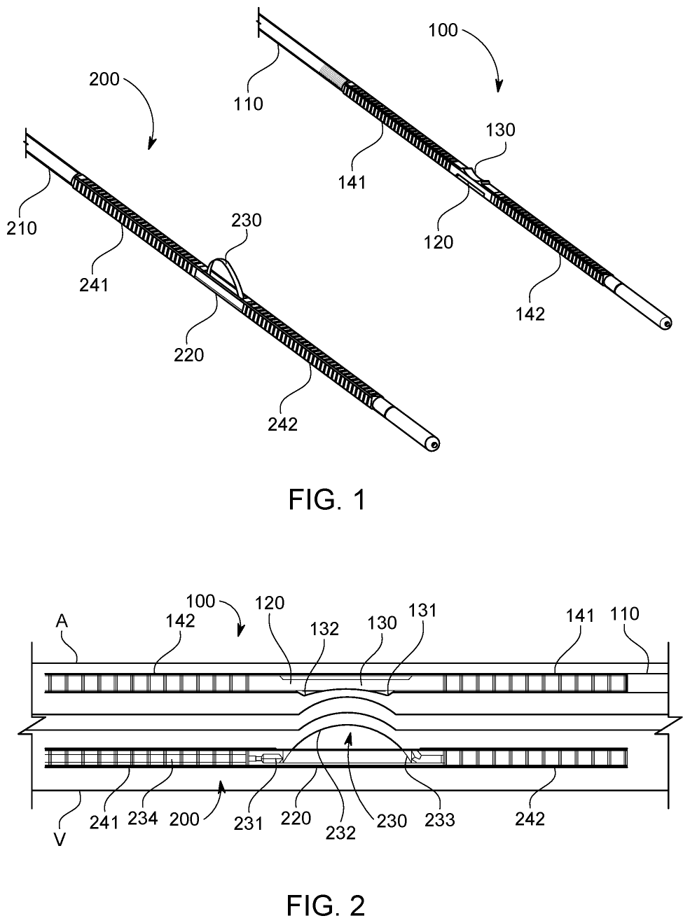

FIG. 1 shows an example of a catheter system for forming a fistula. The system comprises a first catheter 100 and a second catheter 200 which can be used together to form a fistula between two vessels.

The first catheter 100 comprises a catheter shaft 110 and a backstop 120 having a recessed portion 130 disposed at the distal end of the shaft 110. The first catheter 100 may further comprise a proximal set of magnets 141, disposed proximally of the backstop 120, and/or a distal set of magnets 142, disposed distally of the backstop 120.

The second catheter 200 also comprises a catheter shaft 210 and a housing 220 disposed at the distal end of the shaft 210. The housing 220 has an opening and an electrode 230 which is partially disposed in the housing 220 and has a convex portion which extends out of the opening. The housing 220 may be made from a non-conductive ceramic material which can withstand the heat and plasma generated by the electrode 230. The second catheter 200 may also have a proximal set of magnets 241 and/or a distal set of magnets 242 which are disposed proximally and distally of the housing 220, respectively.

The backstop 120 of the first catheter 100 may also be made from a non-conductive ceramic material to withstand the heat and plasma generated by the electrode 230. The recessed portion 130 of the backstop 120 has a concave shape for engaging with the complimentary-shaped convex portion of electrode 230.

FIG. 2 shows the first catheter 100 disposed in an artery A and the second catheter 200 disposed in an adjacent vein V, prior to forming an arteriovenous fistula.

As illustrated in FIG. 2, the backstop 120 of the first catheter 100 includes the recessed portion 130 which has a concave shape. The recessed portion 130 is positioned between a proximal protrusion 131 and a distal protrusion 132 of the backstop 120.

The electrode 230 of the second catheter 200 includes a proximal portion 231, an intermediate portion 232 and a distal portion 233. A connecting element 234 is connected to the proximal portion 231 and extends through the shaft 210 of the second catheter 200. The proximal end of the connecting element 234 may be connected to an RF energy source, for example an ESU pencil, to allow RF energy to be supplied to the electrode 230. The proximal portion 231 may be fixed to the housing 220, for example, with a clamping mechanism or an adhesive. The intermediate portion 232 has a convex shape which extends out of the opening of the housing 220 and away from the housing 220 to contact the vessel wall for forming the fistula. The distance between the top of the intermediate portion 232 and the housing 220 is the height of the electrode 230. The distal portion 233 is not fixed to the housing 220 and can move longitudinally relative to the housing 220. This allows the electrode 230 to move between a contracted configuration, where the electrode 230 is radially contracted, and an expanded where configuration, the electrode 230 extends radially further from the housing 220 than in the contracted configuration.

The electrode 230 may be in the form of a ribbon wire and may be made from a number of suitable materials, such as one or more refractory metals. For example, the electrode 230 may comprise tungsten, molybdenum, niobium, tantalum, rhenium, or combinations and alloys thereof. The housing 220 may be made from a non-conductive ceramic material which can withstand the heat and plasma generated by the electrode 230.

In order to form a fistula between two vessels, such as artery A and vein V, the first catheter 100 is introduced into the arterial system through an access site and advanced to the treatment site where a fistula is to be formed. The second catheter 200 is introduced into the venous system through a second access site and also advanced to the treatment site where the fistula is to be formed. The first catheter 100 and the second catheter 200 may be advanced to the treatment site inside a sheath. The sheath may compress the electrode 230 of the second catheter 200 such that it is in the contracted configuration. This may allow the second catheter 200 to more easily advance through the vessel due to the lower profile. The sheath may then be removed once the first catheter is in position at the treatment site which causes the electrode to move to the expanded position shown in FIG. 2.

The first catheter 100 and second catheter 200 may be advanced to the treatment site from opposite directions, as shown in FIG. 2, or alternatively, they may also be advanced from the same direction.

With the first catheter 100 and the second catheter 200 being introduced from opposite directions, once the first catheter 100 and the second catheter 200 are positioned at the treatment site, the proximal set of magnets 141 of the first catheter 100 will be attracted to the distal set of magnets 242 of the second catheter 200 and align themselves with each other. Similarly, the distal set of magnets 141 of the first catheter 100 will be attracted to the proximal set of magnets 241 of the second catheter 200 and these sets of magnets will align with each other. This will result in the electrode 230 becoming aligned with the recessed portion 130 of the backstop 120. The backstop 120 is configured to compress the vessel walls in a localised region for ablation by the electrode 230 of the second catheter 200. The sets of magnets may also have the effect of pulling the artery A and vein V closer together.

Radiofrequency (RF) energy may then be supplied to the electrode 230 which causes the electrode 230 to heat up generate a plasma. The plasma causes rapid dissociation of molecular bonds in organic compounds and allows the electrode 230 to cut through the venous and arterial vessel walls until it hits the recessed portion 130 of the backstop 120 to form the fistula.

However, the backstop 120 of the first catheter 100 is rigid and this therefore can make it difficult to advance the first catheter 100 past bends in a tortuous vessel to reach the treatment site.

The catheters according to the present disclosure provide a solution to this problem.

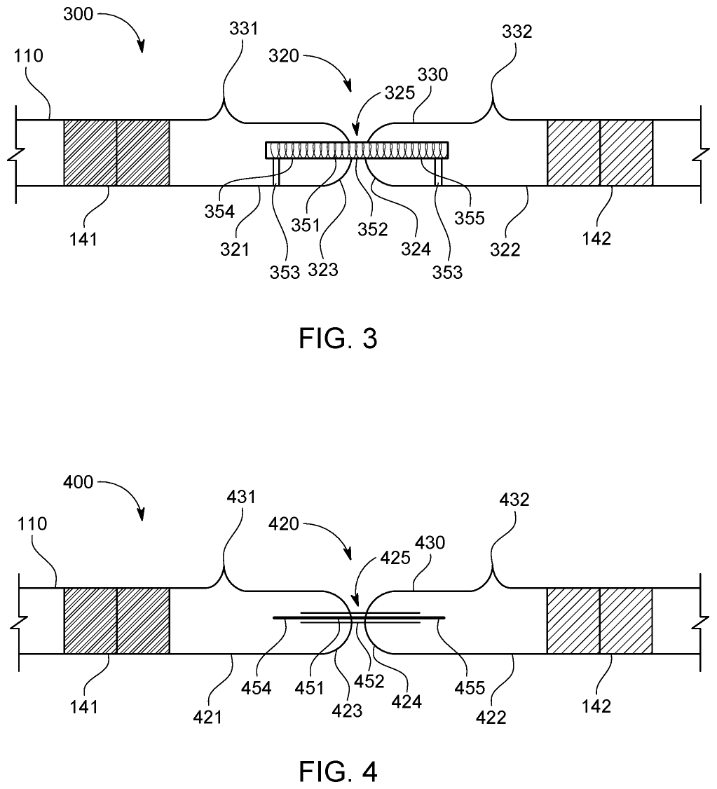

FIG. 3 shows a cross-sectional side view of a distal portion of a first catheter 300. The same reference numerals will be used throughout this disclosure to denote features which are identical across different embodiments.

The first catheter 300 comprises a catheter shaft 110 and a backstop 320 coupled to the catheter shaft 110. Similar to first catheter 100, the backstop 320 may comprise a recessed portion 330 which is defined between a proximal protrusion 331 and a distal protrusion 332. The recessed portion 330 may be substantially U-shaped for engaging the convex portion of the electrode 230. Furthermore, a proximal set of magnets 141 may be disposed proximally of the backstop 320 and a distal set of magnets 142 may be disposed distally of the backstop 320.

The backstop 320 is split into a proximal section 321 and a distal section 322 which are connected such that they are moveable relative to each other. Both the proximal section 321 and the distal section 322 may be made from a non-conductive ceramic material. In the embodiment of FIG. 3, the proximal section 321 and distal section 322 are connected with a coil spring 351. The coil spring 351 may be covered with an insulating material 352. The proximal section 321 may comprise a bore 354 and the distal section 322 may comprise a bore 355 for accommodating the spring 351 and the insulating material 352. A proximal end of the spring 351 may be accommodated within the bore of the proximal section 321 and fixed to the proximal section 321 with an adhesive.

Similarly, a distal end of the spring 351 may be accommodated within the bore of the distal section 322 and fixed to the distal section 322 with an adhesive. In order to make it easier to apply the adhesive, the proximal section 321 and the distal section 322 may each comprise a channel 353 which connects the end of each bore to the outside surface of the backstop 320. An adhesive may be injected into the bore though the channel 353 to attach the end of the spring 351 to the respective ends of the bore of the proximal section 321 and distal section 322.

The first catheter 300 therefore provides a backstop 320 with a degree of flexibility. By splitting the backstop 320 into a proximal section 321 and a distal section 322 and connecting them with a flexible element, such as coil spring 351, the distal section 322 may move relative to the proximal section 321 and vice versa. Specifically, this allows the distal and proximal sections 321, 322 to be tilted relative to each other such that they are disposed at an angle. This way, the first catheter 300 can more easily navigate past bends in a blood vessel to advance to the treatment site.

The spring 351 may be made from a number of suitable materials which provide the required flexibility, such as nitinol, stainless steel or a polymer such as PTFE or HDPE. The insulating material 352 may be in the form of a tube and surround the spring 351 to protect it from the heat and plasma generated by the electrode 230. The insulating material 352 may also prevent any arcing between the electrode 230 and the spring 351, in embodiments where the spring 351 is made from a conductive material. Suitable materials for the insulating material 351 may include polyimide, PTFE, HDPE or PVC, for example.

In the embodiment of FIG. 3, a distal end 323 of the proximal section 321 and a proximal end 324 of the distal section 322 have a rounded shape. These rounded ends allow the proximal and distal sections 321, 322 to tilt to a greater degree with respect to each other, as the edges of the proximal and distal sections 321, 322 will not impede the tilting motion. Furthermore, there may be a small gap 325 between the proximal section 321 and the distal section 322 such that they are not in contact with each other. Again, this will provide a greater degree of flexibility to the backstop 320 and allow for a greater degree of tilt.

The first catheter 300 may be used together with the second catheter 200 to form a fistula, in the same manner as described with respect to FIG. 2 above.

FIG. 4 shows a side view of another embodiment of a first catheter 400.

The first catheter 400 comprises a catheter shaft 110 and a backstop 420, and may further have a proximal set of magnets 141, and a distal set of magnets 142.

The backstop 420 is similar to backstop 320 in that it is split into a proximal section 421 and a distal section 422 which are moveable relative to each other. In the embodiment of FIG. 4, a leaf spring 451 connects the proximal section 421 with the distal section 422. The leaf spring 451 may be encased within an insulating material 452. The leaf spring 451 is fixed to the proximal section 421 and the distal section 422, for example, with an adhesive or a clamping mechanism. The proximal section 421 may comprise a bore 454 and the distal section 422 may comprise a bore 455 for accommodating a proximal end and a distal end of the leaf spring 451, respectively. This arrangement allows the proximal section 421 and distal section 422 to tilt relative to each other and provides flexibility to the backstop 420 such that it can more easily navigate around bends in the blood vessel anatomy.

The proximal section 321 and the distal section 322 may be made from a non-conductive ceramic material. The leaf spring 451 may be made from any suitable flexible material, such as nitinol or tungsten, and may be in the form of a ribbon, for example. The insulating material 452 may be a polyimide, PTFE, HDPE or PVC, for example, which provide good protection for the spring from the heat and plasma generated by the electrode 230 and also prevents arcing between the spring 451 and the electrode 230.

Similar to first catheter 300, the backstop 420 of first catheter 400 may also comprises a proximal protrusion 431 and a distal protrusion 432 which define a central recessed portion 430. The recessed portion 430 may be substantially U-shaped. A distal end 423 of the proximal section 421 and a proximal end 424 of the distal section 422 may also be rounded to provide for a greater degree of tiltability between the proximal section 421 and the distal section 422. Furthermore, there may be a small gap 425 between the proximal section 421 and the distal section 422 such that they are not in contact with each other.

The first catheter 400 may also be used together with second catheter 200 to form a fistula in the same manner as described with respect to FIG. 2, above.

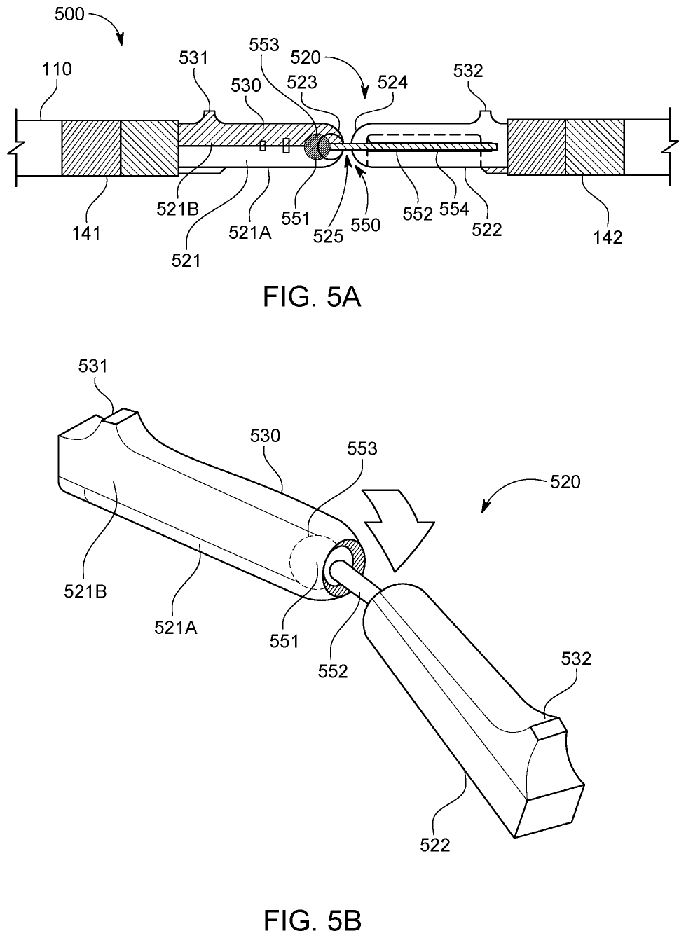

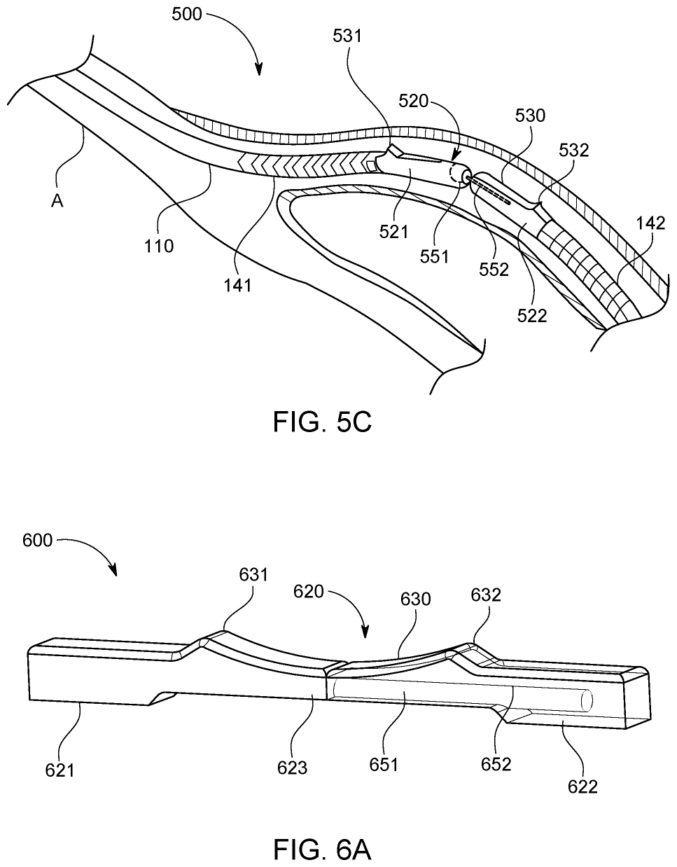

FIG. 5A shows a side view of another embodiment of a first catheter 500.

The first catheter 500 comprises a catheter shaft 110 and a backstop 520, and may further include a proximal set of magnets 141 and a distal set of magnets 142. The backstop 520 may have a recessed portion 530 which is defined between a proximal protrusion 531 and a distal protrusion 532. The recessed portion 530 may be substantially U-shaped for engaging the convex portion of the electrode 230.

The backstop 520 is split into a proximal section 521 and a distal section 522 which are connected through a ball and socket joint 550. The ball and socket joint 550 may comprise a ball 551 which is seated within a spherical socket 553 of the proximal section 521, and a shaft 552 which is secured to the ball 551 at one end and fixed to the distal section 522 at the other end. The distal section 522 may comprise a bore 554 for accommodating the shaft 552. The ball 551 can rotate within the spherical socket and thereby allows the distal section 522 to tilt relative to the proximal section 521, and vice versa, to allow the first catheter 500 to more easily navigate past bends in the blood vessel anatomy.

The proximal and distal sections 521, 522 may be made from a non-conductive ceramic material. Similarly, the ball 551 and the shaft 552 may also be made from a non-conductive ceramic material or a polymer material, for example, PEEK or Nylon. The ball 551 and the shaft 552 may also be coated with an insulating material such as polyimide, PTFE, HDPE or PVC.

The proximal section 521 may be split longitudinally into a first portion 521A and a second portion 521B. When assembling the backstop 520, the first portion 521A and the second portion 521B can be separated to allow the ball 551 to be inserted into the socket formed between the first portion 521A and the second portion 521B. As shown by FIG. 5A, a distal end 523 of the proximal section 521 and a proximal end 524 of the distal section 522 may also be rounded to provide for a greater degree of tiltability between the proximal section 521 and the distal section 522. Furthermore, there may be a small gap 525 between the proximal section 521 and the distal section 522 such that they are not in contact with each other, which therefore allows the proximal and distal sections 521, 522 to tilt to a greater degree relative to each other.

The first catheter 500 may also be used together with second catheter 200 to form a fistula, in the same manner as described with respect to FIG. 2.

FIG. 5B shows an isometric view of the backstop 520 of first catheter 500, where the distal section 522 is tilted relative to the proximal section 521. Rotation of the ball 521 within the spherical socket 553 of proximal section 521 allows the proximal section 521 and the distal section 522 to tilt relative to each other such that they are disposed at an angle relative to each other whilst maintaining a connection through the ball 551 and shaft 552.

FIG. 5C shows the first catheter 500 disposed within an artery A. When the first catheter 500 is introduced into a blood vessel, such as artery A, and advanced to a treatment site, the first catheter 500 will often need to navigate past bends and curves in the blood vessel to reach the treatment site, especially if the fistula is to be formed below the knee, where bends in the blood vessel anatomy can be 90 degrees or more.

FIG. 5C illustrates an exemplary bend in the artery A and shows how, due to the backstop 520 being split into the proximal and distal portions 521, 522 and the ball and socket joint 550 (FIG. 5A), first catheter 500 can more easily navigate this bend. The ball and socket joint 550 allows the proximal section 521 and the distal section 522 to tilt relative to each other such that the backstop 520 can adapt to the shape of the bend and does not become stuck. This equally applies to any other vessel.

First catheters 300 and 400 of FIG. 3 and FIG. 4, respectively, function on the same principle. The spring 351, 451 which connects the proximal section 321, 421 with the distal section 322, 422, is flexible and allows the proximal section 321, 421 to tilt relative to the distal section 322, 422, and vice versa, so that the backstop 320, 420 can better adapt to the shape of the bend of the blood vessel and does not become stuck.

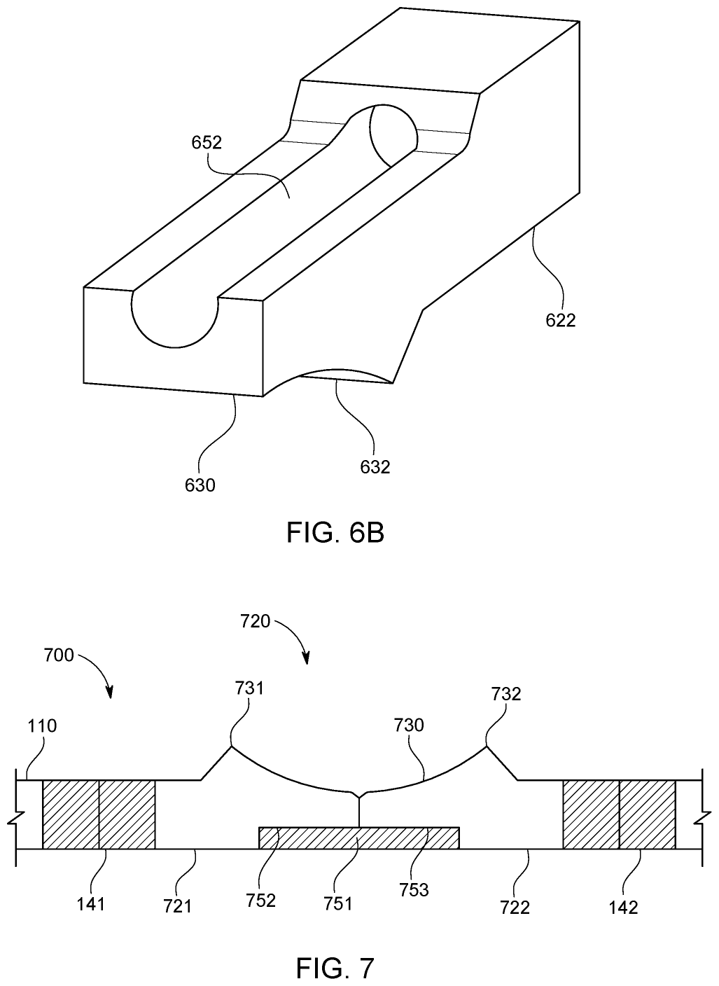

FIG. 6A illustrates another alternative embodiment of a backstop 620 for a first catheter 600. The first catheter 600 may, in addition to the backstop shown in FIG. 6A, comprise a catheter shaft 110, a proximal set of magnets 141 and a distal set of magnets 142, for example.

The backstop 620 is split into a proximal section 621 and a distal section 622. For ease of illustration, the distal section 622 is depicted as being transparent. A proximal protrusion 631 may be disposed on the proximal section 621 and a distal protrusion 632 may be disposed on the distal section 622 which define between them a recessed portion 630. This recessed portion may be convex or U-shaped for engaging with the electrode 230 of the second catheter 200. The proximal section 621 and the distal section 622 may be made from a non-conductive ceramic material, for example.

The proximal section 621 and the distal section 622 are connected through a connecting rod 651. The connecting rod 651 may be in the form of a polyimide tube or an insulated nitinol wire, for example. A polyimide tube provides some flexibility to allow the proximal section 621 and the distal section 622 to tilt relative to each other. Furthermore, the polyimide tube provides good resistance to the heat and plasma generated by the electrode 230 during the fistula forming process. An insulated nitinol wire may have a shape-memory effect and provide sufficient flexibility to allow the proximal section 621 and the distal section 622 to tilt relative to each other. The nitinol wire may be insulated to protect the nitinol wire from the heat and plasma generated by the electrode 230 and also prevent any arcing between the nitinol wire and the electrode 230. The insulating material may be a polyimide, PTFE, HDPE or PVC material, for example.

The connecting rod 651 may be fixed to a distal end 623 of the proximal section 621, for example, with an adhesive. The distal section 622 may comprise a longitudinal channel 652 for accommodating the connecting rod 651. The connecting rod 651 may be secured within the channel 652, for example, with an adhesive. The addition of the channel 652 provides for a more secure fit between the connecting rod 651 and the distal section 622. The surface of the connecting rod 651 and the surface of the channel 652 may be roughened before an adhesive is applied to ensure a good bond between the connecting rod 651 and channel 652.

An advantage of the backstop 620 is that the connecting rod 651 can be made with a smaller diameter which allows a lower profile for the backstop 620 to be achieved. This may be advantageous when trying to move the first catheter 600 through a smaller diameter blood vessel. In the embodiment of FIG. 6A, the proximal section is shown as being in 621 contact with the distal section 622 such that there is no gap. This means that the degree that the proximal section 621 and the distal section 622 can tilt relative to each other is reduced compared to first catheters 300, 400 and 500. However, the structural stability of the backstop 620 is strengthened whilst still allowing for a degree of bending or tilting between the proximal section 621 and the distal section 622 to allow the first catheter 600 to more easily navigate past bends in the blood vessel anatomy.

The first catheter 600 including backstop 620 may be used together with second catheter 200 to form a fistula in the same manner as described with respect to FIG. 2 above.

FIG. 6B shows an isometric view of the distal section 622 of the backstop 620. As illustrated, the channel 652 is machined into the surface of the distal section 622, on the opposite side of the distal section 622 relative to the recessed portion 630. The connecting rod 651 can then be easily accommodated within the channel 652 and fixed to the distal section 622 with an adhesive, for example.

FIG. 7 illustrates another alternative embodiment of a first catheter 700. The first catheter 700 comprises a catheter shaft 110 and a backstop 720, and may further include a proximal set of magnets 141 and a distal set of magnets 142. The backstop 720 may also have a recessed portion 730 which is disposed between a proximal protrusion 731 and a distal protrusion 732. The recessed portion 730 may be concave or U-shaped for engaging the convex portion of the electrode 230.

The backstop 720 is split into a proximal section 721 and a distal section 722 which are connected through a connecting rod 751. The proximal section 721 and the distal section 722 may be made from a non-conductive ceramic material, for example. The connecting rod 751 may be made from the same materials as connecting rod 651 (FIG. 6A), e.g. a polyimide tube or an insulated nitinol wire. The proximal section 721 may comprise a channel 752 which is machined into the surface of the proximal section 721, opposite the recessed portion 730. Similarly, the distal section 722 may comprise a channel 753 which is machined into the surface of the distal section 722 opposite the recessed portion 730. The channels 752 and 753 accommodate the connecting rod 751 which may be secured within the channels 752 and 753 with an adhesive. This allows for a more secure fit between the connecting rod 751 and the proximal and distal sections 721, 722.

Each of the proximal section 721 and the distal section 722 may be identical to the distal section 622 shown in FIG. 6B. The proximal section 721 and the distal section 722 may be in contact with each other to improve the structural stability of the backstop 720.

The split backstop 720 together with the connecting rod 751 allows the proximal section 721 and the distal section 722 to tilt relative to each other which allows the first catheter 700 to better maneuver past bends in the blood vessel anatomy.

Various modifications will be apparent to those skilled in the art.

In any of the embodiments described in this disclosure, the backstop is not limited to a non-conductive ceramic material but may be made from any suitable material which can withstand the heat generated by the electrode.

The backstop of any the above-described embodiments may not have a recessed portion. The backstop may also not have a proximal protrusion or a distal protrusion. Rather, the backstop may comprise another suitably shaped portion for engaging with the electrode. For example, the backstop may comprise a protruding portion.

The recessed portion of the backstop of any of the above-described embodiments is also not limited to a concave or U-shaped portion. The recessed portion may have any suitable shape, such as, for example, a V-shaped portion or a rectangular-shaped portion.

The first catheter of any of the embodiments described herein may not have a set of proximal magnets 141 and/or distal magnets 142.

For any of the backstop embodiments described herein, the distal end of the proximal section may not have a rounded shape

For any of the backstop embodiments described herein, the proximal section of the distal section may not have a rounded shape.

For any of the backstop embodiments described herein, the proximal section and the distal section may be in contact with each other, or there may be a small gap between the proximal section and the distal section such that they are not in contact with each other.

For first catheter 300, the spring 351 may not be insulated.

The proximal section 321 and/or the distal section 322 may not comprise a bore for accommodating the spring 351. Rather, for example, the spring 351 could be attached to the ends of the proximal and distal sections 321, 322.

The spring 351 may be made from any suitable material.

The first catheter 300 may not comprise any channels 353.

For first catheter 400, the spring 451 may not be insulated.

The spring 451 may be made from any suitable material.

The spring 451 could be attached to the ends of the proximal and distal sections 421, 422.

For first catheter 500, the ball 551 and the shaft 552 are not limited to being made from a ceramic material but can be made from any suitable material which can withstand the heat from the electrode.

The proximal section 521 may not be made from two portions 521A, 522B but may rather be formed as a single portion.

The arrangement of the ball and socket joint 550 may be reversed such that the distal section 522 comprises the socket for accommodating the ball 551.

For first catheter 600, the connecting rod 651 may be made from any suitable material and is not limited to polyimide or nitinol.

The arrangement of the backstop 620 may be reversed such that the proximal section 621 comprises a channel for accommodating the connecting rod 651.

The distal section 622 may not have a channel 652. Rather, the connecting rod 651 could be attached to the surface of the distal section 622.

For first catheter 700, the connecting rod 751 may be made from any suitable material and is not limited to polyimide or nitinol.

The proximal section 721 and/or distal section 722 may not have a channel 752, 753. The connecting rod 751 could, for example, be attached to the surface of the proximal and/or distal sections 721, 722.

The electrode 230 of the second catheter 200 is not limited to having a convex shaped portion. The electrode may take any shape suitable for forming a fistula such as, for example, a V-shape or rectangular shape.

The electrode 230 may not be in the form of a ribbon wire but can take any other suitable form which allows the electrode to from a fistula.

The housing 220 may not be made from a ceramic material but can be made for any other suitable material which can withstand the heat of the electrode.

The second catheter 200 may not have a set of proximal magnets 241 and/or distal magnets 242.

All of the above are fully within the scope of the present disclosure and are considered to form the basis for alternative embodiments in which one or more combinations of the above described features are applied, without limitation to the specific combination disclosed above.

In light of this, there will be many alternatives which implement the teaching of the present disclosure. It is expected that one skilled in the art will be able to modify and adapt the above disclosure to suit its own circumstances and requirements within the scope of the present disclosure, while retaining some or all technical effects of the same, either disclosed or derivable from the above, in light of his common general knowledge in this art. All such equivalents, modifications or adaptations fall within the scope of the present disclosure.

1. A catheter for use in a system for forming a fistula between two vessels, the catheter comprising:
a catheter shaft; and
a split backstop coupled to the catheter shaft and configured to receive a fistula-forming electrode, the split backstop having a proximal section and a distal section,
wherein the proximal section and the distal section are moveable relative to each other.

2. The catheter of claim 1, wherein the proximal section and distal section are tiltable relative to each other.

3. The catheter of claim 1, wherein the proximal section and the distal section together form a recessed portion.

4. The catheter of claim 3, wherein the recessed portion has a shape complimentary to the fistula forming electrode.

5. The catheter of claim 3, wherein the recessed portion has a U-shape or, preferably, a concave shape.

6. The system of any of claim 1, wherein the split backstop is non-conductive.

7. The catheter of claim 1, wherein the split backstop is, at least partly, made from a ceramic material.

8. The catheter of claim 1, wherein a distal end of the proximal section has a rounded shape.

9. The catheter of claim 1, wherein a proximal end of the distal section has a rounded shape.

10. The catheter of claim 1, wherein the proximal section and the distal section are connected through a spring.

11. The catheter of claim 10, wherein the spring is insulated.

12. The catheter of claim 10, wherein the proximal section and/or the distal section comprises a bore for accommodating the spring.

13-15. (canceled)

16. The catheter of claim 1, wherein the proximal section and the distal section are connected through a ball and socket joint.

17. The catheter of claim 16, wherein the ball and socket joint comprises a ball disposed within a socket of the proximal section or the distal section, and a shaft attached to the ball.

18. The catheter of claim 17, wherein the ball and shaft are made from a ceramic material.

19. The catheter of claim 17, wherein the ball and shaft are coated with an insulating material.

20. (canceled)

21. The catheter of claim 1, wherein the proximal section and the distal section are connected through a connecting rod.

22. The catheter of claim 21, wherein the proximal section and/or the distal section comprises a bore for accommodating the connecting rod.

23-24. (canceled)

25. The catheter of claim 21, wherein the connecting rod comprises a polyimide tube or a nitinol wire.

26-29. (canceled)

30. A system for forming a fistula, the system comprising:
a first catheter according to any preceding claim; and
a second catheter comprising a housing and a fistula-forming electrode extending radially from the housing.

31-39. (canceled)
